(19) United States
(12) Patent Application Publication
(10) Pub. No.: US 2024/0307280 A1
(43) Pub. Date: Sep. 19, 2024

(54) Homeopathic all natural skin and wound health cleanser and method of use

(71) Applicant: Al E. Roberts, Jacksonville, FL (US)

(72) Inventor: Al E. Roberts, Jacksonville, FL (US)

(21) Appl. No.: 18/121,751

(22) Filed: Mar. 15, 2023

(51) Int. Cl. A61K 8/36; A61K 8/19; A61K 8/92; A61Q 17/00; A61Q 19/10

(52) U.S. Cl. CPC A61K 8/36; A61K 8/19; A61K 8/922; A61Q 17/005; A61Q 19/10; A61K 2800/805

(57) ABSTRACT

A homeopathic, all-natural formulation to treat skin and wound care has been formulated. It will treat all types of wounds but especially skin care wounds. The formulation is effective in the reduction of biofilm that may promote infection and the formulation is non-toxic and can be applied either in a liquid form or a gel form. The formulation uses a specific combination of all natural ingredients that are combined in a specific ratio to achieve the desired result. The frequency of application of the formula is determined by someone trained in the medical arts in this field.

### BACKGROUND OF THE INVENTION

### A. Field of the Invention

This solution that will be applied to the skin and areas of wound health. It combines well-known ingredients in a very specific ratio to form an all-natural acidic pH formulation that is ideal for wound care for skin. The solution can reduce biofilm while at the same time promoting healthy growth within and around the area of the wound. It is meant to be applied on the skin either in a liquid form or a gel form and is not harmful to the skin tissue.

### B. Prior Art

There are prior art references to applications that promote or tend to promote homeopathic remedies for wound treatment.

A representative example of this is found at Dovlatavadi, U.S. Pat. No. 10,905,729. This uses a water extract of dried leaves with water. The solution that is used in this particular application is stable for at least 28 hours for the topical treatment of wounds. It has been stable for up to 2 years in clinical testing. It is applied to the surface of the wound using a spray followed by the application of extract soap and gauze dressing to the surface of the wound.

Another representative example in the prior art can be found at Arkin, U.S. Patent publication 2005/0137164. The Arkin reference teaches pharmaceutical compositions and methods for the treatment of skin disease and disorders such as actinic keratosis. This is an applied gel of a non-steroidal anti-inflammatory drug such as Diclofenac.

Other representing examples include Poritopoulos, Patent publication 2021/0244029; Nobile, U.S. Patent publication 2022/0110897; and Dobmever, U.S. Patent publication 2022/0160649.

While all the ingredients in this application are common and are used in the health care industry for healing purposes, none of the other prior art references use the ingredients in the specific ratios for healing purposes.

### BRIEF SUMMARY OF THE INVENTION

The skin is the largest organ of the body and is subject to many different disorders and diseases, including skin cancer, rashes, and infection that can be caused by a variety of ailments including diabetes. The human body has remarkable recuperative powers dealing with injuries and in particular injuries to skin. The types of wounds that are suffered by skin are numerous and the types of care for those wounds is equally many and varied.

A key component to effective skin wound care is the reduction of biofilm on the wound itself. Biofilm is a collective of one or more types of microorganisms that can grow on the surface of a wound. Microorganisms that form biofilm include bacteria, fungi, and extracellular polymeric secretions. Another common example of biofilm is dental plaque, which is a slimy buildup of bacteria that forms on the surface of teeth.

Biofilms have numerous harmful effects such as the growth and lingering effects of infections. It is the reduction of this biofilm that will improve the recovery of the person who has suffered a skin, wound, injury. It is important in wound care to reduce the biofilm on the wound itself while at the same time enhancing and accelerating the healing processes. These beneficial effects are accomplished with this formulation that will be dispensed in either a liquid form or gel form. This formulation combines natural ingredients that when combined eradicate the growth of biofilm and lead to healing.

The ingredients that are used with this formulation include the following: distilled water, acetic acid, glycerin, sodium bicarbonate, and lavender oil. The formulation for making 1000 ml of the solution is 780 ml of distilled water, 200 ml of 5% acetic acid, 20 ml of glycerin, 5 ml of sodium bicarbonate, and 0.5 ml of lavender oil. Acetic acid is an acidic, colorless liquid; vinegar is at least four percent acetic acid by volume and acetic acid is the main component of vinegar apart from water. Glycerin is soap that is added to the formulation because of its general healing properties. Sodium bicarbonate is a white, alkaline chemical compound, in powder form, that is used to achieve the desired pH of the formula. The lavender oil adds additional healing properties to the formula and gives the solution a pleasant order while at the same time deflecting or diluting some of the vinegar smell from the acetic acid.

In addressing wound care it is important to maintain the pH of the wound; the pH (power of hydrogen) measure is a common measure of the acidity or alkalinity of a liquid or environment. An environment with a pH of less than 7 is considered acidic and an environment with a pH of more than 7 is considered alkaline. The human body maintains a skin pH of approximately 5.5 and wounds generally have a pH in a range of between 4 and 6. In wound care it is important to maintain the pH of the wound of between 4 and 5 (acidic) to promote rapid healing.

The formulation encourages the pH of the wound area be maintained at the desired range for fast and effective healing.

### BRIEF DESCRIPTION OF THE DRAWINGS

No drawings are needed in this application.

### DETAILED DESCRIPTION OF THE EMBODIMENTS

For purposes of this application the care of a diabetic foot wound will be highlighted. If the disease of diabetes is left unchecked, it can have devastating effects on many parts of the human body including skin. The effects of diabetes can produce ulcers on the feet that must be treated. If the ulcers are left untreated, the ulcers will continue to degrade, and can result in even greater damage.

The cleanser consists of the following ingredients: distilled water, five percent acetic acid, glycerin, sodium bicarbonate, and lavender oil. The formulation is made with the ingredients in the following percentages for a 1000 ml of the cleanser: 780 ml of distilled water, 200 ml of 5% acetic acid, 20 ml of glycerin, 5 ml of sodium bicarbonate, and 0.5 ml of lavender oil.

In the case of a diabetic ulcer on the skin, particularly the foot, it is important to treat the wound to achieve maximum healing in the quickest possible time. After the skin wound is properly debrided, the cleanser is applied to the affected skin area, then the wound is wrapped with an appropriate dressing. The wound must remain wrapped during the healing process. The wound is typically covered for a minimum of twenty-four hours before the wound is again exposed, examined, and retreated. The course of treatment for the wound will vary depending on the type of wound and the professional judgment of the health care professional. Depending on the wound itself, the formulation can be reapplied as often as necessary, and the wound rewrapped with a clean bandage after each inspection and cleaning. The process is repeated until there is sufficient healing of the wound, whereas intact skin is present, or that is determined by the health care professional.

In addition to the liquid formulation, there will also be a gel that can be applied to the affected area in the same manner as the liquid. The formulation can be applied directly to the skin, wound, pad, or gauze.

This formulation is not intended to replace the sound medical decision of the person who is applying the formulation to the affected area including the amount of debridement, or the frequency of application of the formulation.

While the embodiments of the invention have been disclosed, certain modifications may be made by those skilled in the art to modify the invention without departing from the spirit of the invention.

The inventor claims:

1. A homeopathic all natural skin and wound care cleanser, which is comprised of:
a composition which consists of the following:
distilled water,
wherein the distilled water is seventy-eight percent of the formulation,
five percent acetic acid,
wherein the five percent acetic acid is twenty percent of the formulation,
glycerin,
where the glycerin is two percent of the formulation,
sodium bicarbonate,
wherein a predetermined amount of sodium bicarbonate is added to the formulation,
lavender oil,
wherein a predetermined amount of lavender oil is added to the formulation,
wherein the ingredients are combined according to the ratios outlined above into a solution.

2. The homeopathic all natural skin and wound care cleanser as described in claim 1 wherein the solution is applied in a liquid form.

3. The homeopathic all natural skin and wound care cleanser as described in claim 1 wherein the solution is applied in a gel form.

4. A method to apply a homeopathic all natural skin and wound care cleanser, which is comprised of the following steps:
examination of the wound,
debridement of the wound,
application of the formulation to the affected area,
wrapping the affected area,
unwrapping the affected area after a predetermined amount of time,
reapplication of the formulation,
rewrapping the affected area,
repeating the affected area and repeating the previously outlined steps.

5. The method to apply a homeopathic all natural skin and wound care cleanser as described in claim 4 wherein the cleanser is applied directly to the skin or into the wound.

6. The method to apply a homeopathic all natural skin and wound care cleanser as described in claim 4 wherein the cleanser is applied directly to a pad prior to applying it to the wound.

7. The method to apply a homeopathic all natural skin and wound care cleanser as described in claim 4 wherein the cleanser is applied directly to gauze prior to applying it to the wound.
